(19) United States
(12) Patent Application Publication
(10) Pub. No.: US 2020/0170746 A1
(43) Pub. Date: Jun. 4, 2020

(54) BIO-IMPLANTABLE MATERIAL WINDING SYSTEM

(71) Applicant: KAWASUMI LABORATORIES, INC., Oita (JP)

(72) Inventors: Seiichi NAKAYA, Oita (JP); Noriaki SHIRAHAMA, Oita (JP)

(21) Appl. No.: 16/640,367

(22) PCT Filed: Aug. 27, 2018
(86) PCT No.: PCT/JP2018/031562

§ 371 Date: Feb. 20, 2020

(30) Foreign Application Priority Data
Aug. 28, 2017 (JP) 2017-163497

(51) Int. Cl. A61B 90/00

(52) U.S. Cl. CPC A61B 90/08; A61B 2090/0816

(57) ABSTRACT

An objective of the present invention is achieved by a bio-implantable material winding system constituted by a sheet winding system comprising a sheet-shaped bio-implantable material F and a sheet winding device capable of winding the bio-implantable material, said sheet winding device comprising an axle member and a cylindrical member fitted onto the axle member and capable of moving relative to the axle member in the axle direction. The bio-implantable material winding system is configured to be capable of retaining the bio-implantable material in a rolled state in a gap between a leading end part of the axle member and the cylindrical member covering the leading end part. The bio-implantable material is configured to be deployable so as to assume a sheet-like shape when the leading end part of the axle member is exposed and the retaining action by the sheet winding device released.

### TECHNICAL FIELD

The present invention relates to a bio-implantable material winding system.

### BACKGROUND ART

Conventionally, a sheet-like anti-adhesion material for reducing body tissue adhesion that can occur due to surgery or external injury or the like is known.

Also, in recent years, arthroscopic surgery using a laparoscope or the like has been performed, and a medical instrument capable of winding up sheet-like anti-adhesion material in order to insert the anti-adhesion material into the body during such surgery has also be proposed (refer to Patent Document 1 for example).

### PRIOR ART DOCUMENT

### Patent Document

Patent Document 1: Japanese Patent Application Publication No. 2013-66671

### SUMMARY OF THE INVENTION

### Technical Problem

However, in the case of Patent Document 1 described above, a holding portion at the tip end of an operating rod must be made to hold the sheet-like anti-adhesion material in advance, and this is a complicated task. Moreover, when applying the anti-adhesion material to a desired site, if the anti-adhesion material does not expand sufficiently when the hold on the anti-adhesion material is released, the anti-adhesion material will not be able to be suitably applied, and workability will end up deteriorating. That is, in order to improve the handleability of the anti-adhesion material, it is necessary to optimize not only the structure of the device itself that holds the anti-adhesion material and releases that hold, but also the shape and physical properties of the anti-adhesion material.

Note that such a problem is not limited to sheet-like anti-adhesion material, and may also arise in other sheet-like bio-implantable material such as sheet-like tissue filling material, for example.

Therefore, in view of such a problem, it is an object of the present invention to improve the handleability of sheet-like bio-implantable material, and make the application to a desired site easier.

### Solution to Problem

In order to solve the aforementioned problem, one aspect of the present invention relates to

a bio-implantable material winding system provided with sheet-like bio-implantable material, and a winding device capable of winding the bio-implantable material, in which

the winding device comprising:

a shaft member, and

a cylindrical member that fits over the shaft member and is able to move in the axial direction relative to the shaft member, wherein

the winding device is able to hold the bio-implantable material in a rolled shape in a gap between a tip end portion of the shaft member and the cylindrical member that covers the tip end portion, and

the bio-implantable material is able to expand to a sheet-like shape when the tip end portion is exposed and the hold by the winding device is released.

### Advantageous Effect of the Invention

According to the present invention, the handleability of sheet-like bio-implantable material can be improved, and the application to a desired site can be made easier.

### BRIEF DESCRIPTION OF THE DRAWINGS

FIG. 1 is a perspective view of the general configuration of a sheet winding system according to one embodiment the present invention.

FIG. 2 is an exploded perspective view of the sheet winding system in FIG. 1.

FIG. 3A and FIG. 3B are views of a sheet winding device provided in the sheet winding system in FIG. 1.

FIG. 4 is a sectional view taken along the axial direction of the sheet winding device in FIG. 3.

FIG. 5 is a view for explaining how to use the sheet winding system in FIG. 1.

FIG. 6 is a view for explaining how to use the sheet winding system in FIG. 1.

### DESCRIPTION OF THE EMBODIMENT

Hereinafter, an embodiment of the present invention will be described with reference to the drawings.

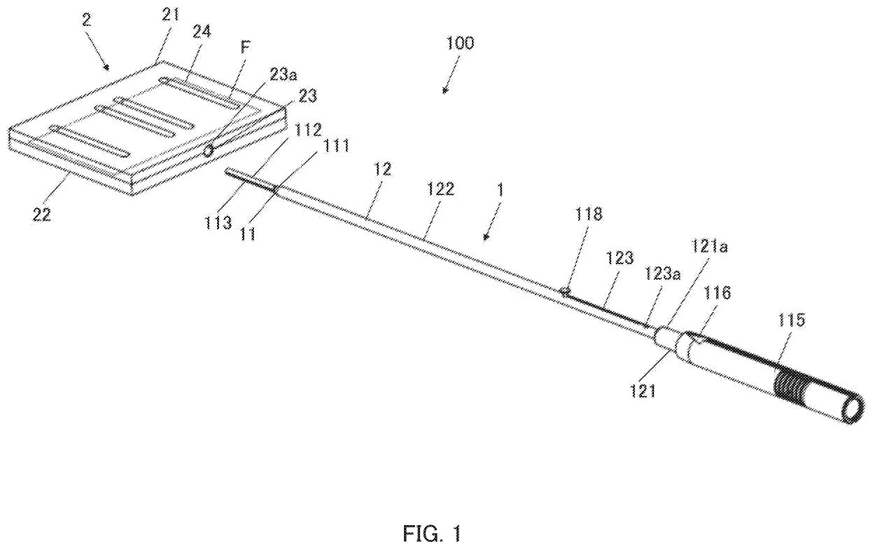

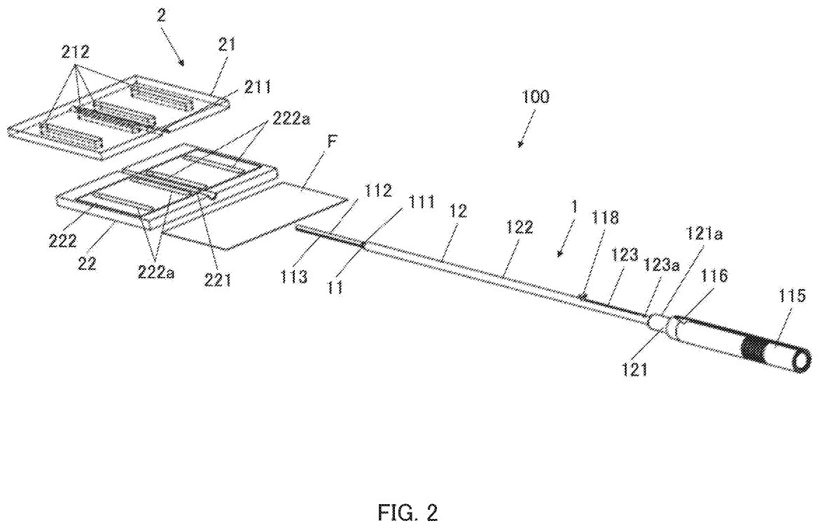

FIG. 1 is a perspective view of the general configuration of a sheet winding system 100 according to one embodiment of the present invention, and FIG. 2 is an exploded perspective view of the sheet winding system 100.

The sheet winding system 100 of the present embodiment is designed to wind sheet-like bio-implantable material F to be inserted into a body, and specifically, is provided with a sheet winding device 1 that winds the bio-implantable material F, and a sheet holder (sheet accommodating device) 2 that holds the bio-implantable material F to be wound by this sheet winding device 1, as illustrated in FIG. 1 and FIG. 2.

Note that in the description below, the axial direction of the sheet winding device 1 is the front-rear direction, one direction orthogonal to the front-rear direction is the left-right direction, and the direction orthogonal to the front-rear direction and the left-right direction is the up-down direction.

Also, the far side (distal side) as viewed from a user gripping the sheet winding device 1 is the tip end side, and the near side (proximal side) as viewed from the user is the base end side.

<Bio-Implantable Material>

First, the bio-implantable material F will be described.

The bio-implantable material F includes anti-adhesion material to be inserted into a body in order to reduce tissue adhesion, for example.

Here, the anti-adhesion material substantially consists of a water-soluble polymer (A) and a poly aliphatic ester (B), and forms a single layer structure of a two-component composition, or forms a laminated structure in which a coating layer substantially consisting of a poly fatty acid ester (B) is formed so as to cover the surface (both sides or one side) of a base layer substantially consisting of a water-soluble polymer (A), for example. Note that the term “substantially” means that a trace amount of impurities that are inevitably mixed in during production and the like may be included, but other components are not intentionally added.

As the water-soluble polymer (A), for example, a polysaccharide, protein, or synthetic polymer or the like can be used, and from the viewpoint of enhancing pliability of the entire anti-adhesion material, it can be particularly preferable to use pullulan.

As the poly aliphatic ester (B), a polylactic acid, a polyglycolic acid, a polycaprolactone and a copolymer thereof or the like can be used because of their excellent in vivo compatibility, and a lactic acid/glycolic acid/ε-caprolactone terpolymer (LA/GA/ε-CLT) having a molecular weight of approximately 20,000 to 300,000 is preferable.

Also, the anti-adhesion material may contain a cell growth inhibitor having an effect of suppressing the growth of cells in contact with the anti-adhesion material when the anti-adhesion material is applied to a wound area in the body. Examples of the cell growth inhibitor include acid, an anticancer agent, a cellular inhibitor, an anti-inflammatory agent, a steroid, an antibacterial agent, and an antibiotic and the like.

Also, the bio-implantable material F has, for example, a sheet-like (film-like) shape having a predetermined thickness, and is formed in a substantially rectangular shape that is wide in the left-right direction.

The dimensions of the bio-implantable material F are, for example, a thickness in the up-down direction of 0.02 to 1 [mm], a length in the front-rear direction of 50 to 150 [mm], and a length in the left-right direction of 50 to 150 [mm], but these are only examples; the dimensions are not limited to these.

Also, in accordance with JIS K6745, the bio-implantable material F preferably has a rigidity index value obtained by dividing the modulus of rigidity [MPa] when measured using a Clash-Berg type flexibility tester (made by Toyo Seiki) by the thickness [mm], of approximately 5,000 to 2,000,000, and more preferably, approximately 600,000 to 1,500,000. For example, when the thickness of the bio-implantable material F is approximately 0.05 [mm], a preferred modulus of rigidity [MPa] is approximately 250 to 100,000, and more a preferred modulus of rigidity [MPa] is approximately 30,000 to 75,000. That is, if the rigidity index value becomes greater than 2,000,000, it becomes difficult to wind the bio-implantable material F into a rolled shape by using the sheet winding device 1, and the bio-implantable material F will not readily expand to the sheet-like shape when the hold is released; on the other hand, if the rigidity index value becomes less than 5,000, the bio-implantable material F will not readily expand to the sheet-like shape when the hold by the sheet winding device 1 is released.

In this way, by adjusting the rigidity index value of the bio-implantable material F to within the range described above, the bio-implantable material F can be said to have a modulus of rigidity that enables it to assume the rolled shape when held by the sheet winding device 1, and to expand to the sheet-like shape when the hold is released.

Note that as the shape of the bio-implantable material F, a rectangular shape is given as an example, but this is only an example and the shape of the bio-implantable material F is not limited to this; for example, the shape of the bio-implantable material F can be arbitrarily changed, as appropriate, to a square shape or a circular shape or the like. Also, as the bio-implantable material F, anti-adhesion material is given as an example, but this is only an example and the bio-implantable material F is not limited to this; for example, the bio-implantable material F can be arbitrarily changed, as appropriate, to tissue filling material or the like.

<Sheet Holder>

Next, a sheet holder 2 will be described.

The sheet holder 2 has an upper holder structure member 21 and a lower holder structure member 22, for example.

More specifically, for example, the upper and lower holder structure members 21 and 22 are placed in an overlapped state by displacing at least one from among the upper holder structure member 21 and the lower holder structure member 22 such that they become relatively closer together, and making the lower surface of the upper holder structure member 21 and the upper surface of the lower holder structure member 22 face each other (refer to FIG. 1). On the other hand, the upper and lower holder structure members 21 and 22 are taken out of the overlapped state by displacing at least one from among the upper holder structure member 21 and the lower holder structure member 22 such that they become relatively farther apart from each other, and separating the lower surface of the upper holder structure member 21 and the upper surface of the lower holder structure member 22 (not shown in the drawings).

Also, the sheet holder 2 has a through-hole 23 (refer to FIG. 1) formed through it in the front-rear direction.

That is, an upper groove 211 having a semicircular cross section is formed countersunk from the front side toward the rear side, on the lower surface of the upper holder structure member 21, as illustrated in FIG. 2. Also, a plurality of (for example, four) through-holes 212 that pass through vertically are provided in the upper holder structure member 21.

A lower groove 221 having a semicircular cross section is formed countersunk from the front side toward the rear side, on the upper surface of the lower holder structure member 22. More specifically, the lower groove 221 in the lower holder structure member 22 is formed in a position facing the upper groove 211 in the upper holder structure member 21 when the upper and lower holder structure members 21 and 22 are overlapped (refer to FIG. 1).

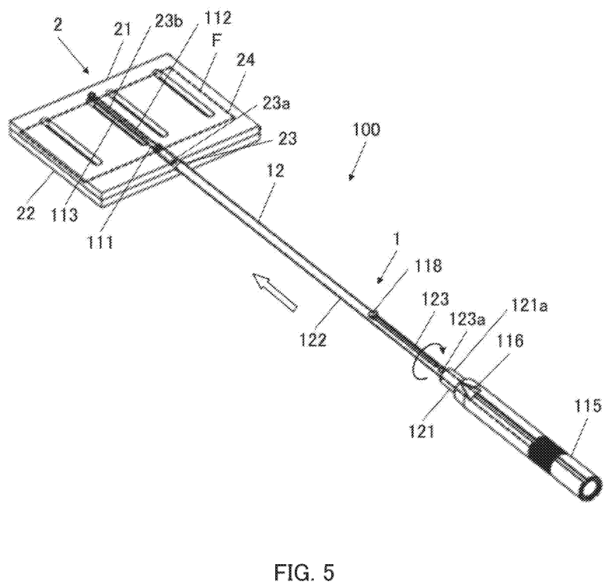

The upper groove 211 and the lower groove 221 are such that the portions on the front side have a larger diameter than the portions on the rear side. Also, the large diameter portions on the front side of the upper groove 211 and the lower groove 221 have substantially the same diameter, and form a large hole 23a having a substantially circular cross section when the upper and lower holder structure members 21 and 22 are overlapped (refer to FIG. 1). Similarly, the small diameter portions on the rear side of the upper groove 211 and the lower groove 221 have substantially the same diameter, and form a small hole (hole) 23b having a substantially circular cross section when the upper and lower holder structure members 21 and 22 are overlapped (refer to FIG. 5 (described later)).

That is, the through-hole 23 is formed by communicating the large hole 23a on the front side with the small hole 23b on the rear side. This through-hole 23 is formed in substantially the center in the left-right direction of the sheet holder 2, but this arrangement of the through-hole 23 is only example; the arrangement is not limited to this.

The inside diameter of the large hole 23a is larger than the outside diameter of a cylindrical member 12 (described in detail later) of the sheet winding device 1 that will be described later.

The inside diameter of the small hole 23b is smaller than the outside diameter of the cylindrical member 12 (described in detail later) of the sheet winding device 1, and is larger than the outside diameter of a tip end portion 112 (described in detail later) of a shaft member 11.

That is, both the tip end portion 112 of the shaft member 11 and the cylindrical member 12 can be removably inserted into the large hole 23a, whereas the tip end portion 112 can be removably inserted into the small hole 23b but the cylindrical member 12 cannot be inserted into the small hole 23b.

Also, the length in the front-rear direction of the small hole 23b is equal to, or longer than, the length in the front-rear direction of the bio-implantable material F.

Note that the edge on the front side of the through-hole 23 (large hole 23a) preferably increases in diameter in a tapered shape to facilitate insertion of the tip end portion 112 of the shaft member 11, for example.

Also, the sheet holder 2 has an accommodating portion 24 that is communicated with the through-hole 23 and accommodates the bio-implantable material F.

That is, a sheet arranging portion 222 is formed on the upper surface of the lower holder structure member 22 by being countersunk a predetermined depth (for example, approximately 0.2 to 0.3 [mm]) in accordance with the thickness of the bio-implantable material F. This sheet arranging portion 222 is formed, for example, continuous with the small diameter portion on the rear side of the lower groove 221, and the accommodating portion 24 that is communicated with the through-hole 23 is formed by the sheet arranging portion 222 being covered by the upper surface of the upper holder structure member 21 when the upper and lower holder structure members 21 and 22 are overlapped (refer to FIG. 1).

Also, the sheet arranging portion 222 is arranged corresponding to the small hole 23b (small diameter portion on the rear side of the lower groove 221), and the length in the front-rear direction of the sheet arranging portion 222 is equal to, or longer than, the length in the front-rear direction of the bio-implantable material F, similar to the small hole 23b.

Also, a plurality of (for example, four) recesses 222a are formed on the upper surface of the sheet arranging portion 222 by being countersunk a predetermined depth.

Note that although not shown in the drawings, the sheet arranging portion 222 may be cut out to the front side of the sheet holder 2, for example, in which case the bio-implantable material F can be placed in the accommodating portion 24 from the front side even when the upper and lower holder structure members 21 and 22 are overlapped (refer to FIG. 1).

Also, the upper and lower holder structure members 21 and 22 of the sheet holder 2 are made of PP, PET, or polycarbonate or the like, for example, but these are only examples; the material of the upper and lower holder structure members 21 and 22 is not limited to these and may be arbitrarily changed as appropriate.

<Sheet Winding Device>

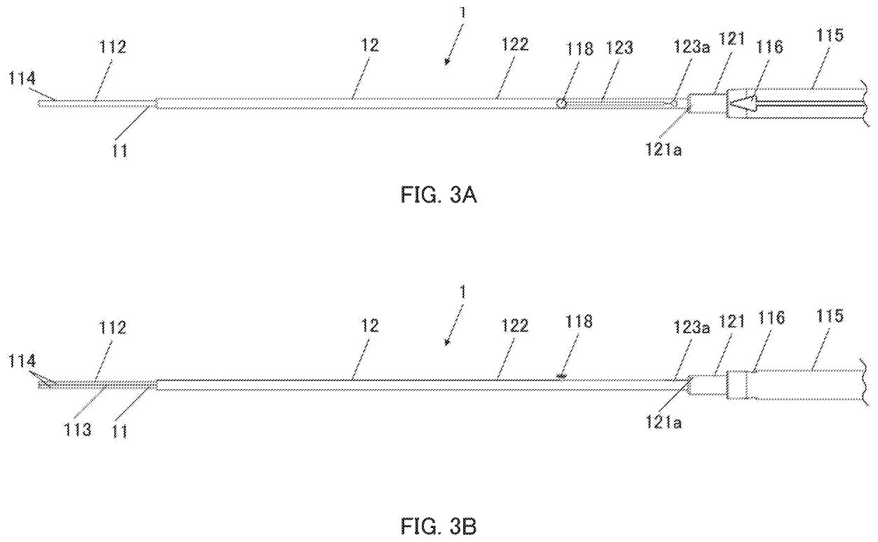

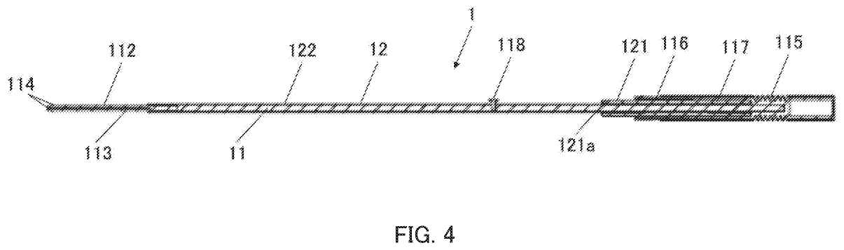

Next, the sheet winding device 1 will be described with reference to FIG. 3A, FIG. 3B, and FIG. 4.

FIG. 3A is a plan view of the sheet winding device 1, and FIG. 3B is a side view of the sheet winding device 1 as viewed from the left side. Also, FIG. 4 is a sectional view taken along the axial direction of the sheet winding device 1.

The sheet winding device 1 is provided with the shaft member 11 and a cylindrical member 12 that is fitted over this shaft member 11, as illustrated in FIG. 3A and FIG. 3B.

The shaft member 11 has, for example, a long cylindrical shaft main body 111, and the tip end portion 112 detachably formed on this shaft main body 111.

The cylindrical portion of the tip end portion 112 that has a smaller diameter than the shaft main body 111 is split into two along the axial direction from the tip end side, such that an inserting slit (inserting portion) 113 into which the bio-implantable material F can be inserted from the tip end side is formed by two members 114 and 114 having substantially semicircular cross sections, for example. Also, the tip end portion 112 has a plurality of these inserting slits 113 of different dimensions (lengths in the front-rear direction, in particular), which can be used interchangeably in accordance with the dimensions (the length in the front-rear direction, in particular) of the bio-implantable material F. Also, the gap of the inserting slit 113 is set, as appropriate, in accordance with the thickness of the bio-implantable material F, and is approximately 0.1 [mm], for example. Also, the diameter when the cross section in the direction orthogonal to the axial direction of the tip end portion 112 is assumed to be substantially circular, is at least approximately 3 [mm]. That is, if the diameter of the tip end portion 112 is too small, then even when the hold by the sheet winding device 1 on the bio-implantable material F that is wound around the tip end portion 112 is released, as will be described later, the bio-implantable material F will not readily expand naturally to the sheet shape.

Also, on the most tip end side of the tip end portion 112, an inclined surface is formed such that the separation distance (gap) increases toward the tip end side so that the bio-implantable material F can easily be inserted into the inserting slit 113.

Note that a detachment inhibiting portion that protrudes radially outward so as to inhibit the bio-implantable material F in a state wound around the tip end portion 112 from detaching from the inserting slit 113 may be formed on the most tip end side of the tip end portion 112.

The outside diameter of the tip end portion 112 is smaller than the inside diameter of the small hole 23b of the through-hole 23 of the sheet holder 2, such that the tip end portion 112 can be inserted into the small hole 23b and can rotate around its axis.

That is, the bio-implantable material F held in the accommodating portion 24 is inserted into the inserting slit 113 by inserting the tip end portion 112, such that the bio-implantable material F and the inserting slit 113 are in the same orientation, into the small hole 23b of the sheet holder 2 in which the bio-implantable material F is held in the accommodating portion 24 (refer to FIG. 5). Also, the bio-implantable material F is wound in a rolled shape around the tip end portion 112 by rotating the tip end portion 112 around its axis while the bio-implantable material F is in a state inserted in the inserting slit 113.

Note that the orientation of the inserting slit 113 refers to the extending direction of the gap between the two members 114 and 114 having substantially semicircular cross sections that form the inserting slit 113 when the tip end portion 112 is viewed from the distal side toward the base end side. For example, when the bio-implantable material F is held horizontally in the sheet holder 2, the bio-implantable material F is inserted into the inserting slit 113 by the tip end portion 112 being inserted into the small hole 23b in a state in which the shaft member 11 has been rotated to an orientation such that the extending direction of the gap between the two members 114 and 114 that have substantially semicircular cross sections becomes horizontal.

A gripping portion 115 to be gripped by the user is connected to the base end side of the shaft main body 111.

The gripping portion 115 is formed having a larger diameter than the shaft main body 111, for example. Also, an indicating portion 116 that correlates with the orientation of the inserting slit 113 and indicates the inserting orientation of the inserting slit 113 with respect to the bio-implantable material F, is provided on the tip end portion of the gripping portion 115. More specifically, the indicating portion 116 is formed by machining the surface of the gripping portion 115 in the shape of an arrow indicating the inserting orientation of the inserting slit 113, for example.

Note that the arrangement of the indicating portion 116 is only an example and is not limited to this; for example, although not shown in the drawings, the indicating portion 116 may be provided in a position other than on the tip end portion of the gripping portion 115. Furthermore, the indicating portion 116 may be provided on a constituent element (such as the shaft main body 111 for example) of the shaft member 11 other than the gripping portion 115, or on a constituent element (such as the cylindrical member 12 for example) of the sheet winding device 1 other than the shaft member 11.

Also, a base end accommodating portion 117 capable of accommodating a large diameter cylindrical portion 121 of the cylindrical member 12 is provided on the tip end side of the gripping portion 115.

That is, the gripping portion 115 is formed, for example, in a cylindrical shape with a bottom, corresponding to the outer shape of the large diameter cylindrical portion 121 of the cylindrical member 12, and the internal space thereof forms the base end accommodating portion 117. Also, the end on the base end side of the shaft main body 111 is connected to substantially the center of the bottom surface of the base end accommodating portion 117. As a result, the large diameter cylindrical portion 121 of the cylindrical member 12 is accommodated in the base end accommodating portion 117 when the cylindrical member 12 is displaced toward the base end side along the axial direction relative to the shaft member 11.

The cylindrical member 12 is a member having an overall cylindrical shape, for example, and has a large diameter cylindrical portion 121 and a cylindrical main body 122 formed continuously with this large diameter cylindrical portion 121.

The large diameter cylindrical portion 121 and the cylindrical main body 122 have an inside diameter that is larger than the outside diameter of the shaft main body 111 of the shaft member 11, and are able to move in the axial direction relative to the shaft member 11, in a state fitted over the shaft member 11.

When the large diameter cylindrical portion 121 is accommodated in the base end accommodating portion 117, for example, the tip end side portion of the large diameter cylindrical portion 121 is exposed. Also, this exposed tip end side portion forms an operation portion 121a that is operated to displace the cylindrical member 12 in the axial direction relative to the shaft member 11. Note that the operation portion 121a may be formed in a shape that makes it easy for the user to catch his or her hand or finger on it, for example.

The cylindrical main body 122 is formed continuous with the tip end side of the large diameter cylindrical portion 121, and the outside diameter of the cylindrical main body 122 is smaller than the outside diameter of the large diameter cylindrical portion 121. Also, the cylindrical main body 122 is formed from flexible material such as resin, for example.

Also, a retaining slit 123 with which a protrusion 118 on the shaft main body 111 engages (external fitting release restricting portion) is provided in the cylindrical main body 122. That is, the length in the front-rear direction (axial direction) of the cylindrical main body 122 is slightly shorter than the length in the front-rear direction of the shaft main body 111, for example, and the long hole-shaped retaining slit 123 that passes through the inside and outside of a tube wall is formed in the base end side portion.

The length (width) in the direction orthogonal to the longitudinal direction of the retaining slit 123 is, for example, equal to or slightly larger than the diameter of a shaft portion of the protrusion 118. Also, an engaging portion 123a that engages with the shaft portion of the protrusion 118 is provided on the base end side portion of the retaining slit 123. That is, the width of a portion slightly to the tip end side of the base end portion of the retaining slit 123 is pinched (made narrower) so as to be smaller than the diameter of the shaft portion of the protrusion 118, and the portion from this pinched portion to the base end portion forms the engaging portion 123a.

As a result, when at least one from among the shaft member 11 and the cylindrical member 12 is moved such that the cylindrical member 12 is displaced toward the tip end portion 112 side relative to the shaft member 11, the shaft portion of the protrusion 118 of the shaft main body 111 becomes engaged with the engaging portion 123a on the base end side of the retaining slit 123, and as a result, movement of the cylindrical member 12 along the axial direction is restricted, and the externally fitted state is restricted from being released.

Also, when at least one from among the shaft member 11 and the cylindrical member 12 is moved such that the cylindrical member 12 is displaced toward the base end side relative to the shaft member 11, the shaft portion of the protrusion 118 of the shaft main body 111 comes into contact with the tip end portion of the engaging portion 123a, and the externally fitted state is restricted from being released. In this state, the entire tip end portion 112 of the shaft member 11 is exposed (refer to FIG. 3A and the like), so the bio-implantable material F is able to be inserted into the inserting slit 113.

Also, the cylindrical main body 122 covers the tip end portion 112 in a state in which the bio-implantable material F is wound, so the bio-implantable material F can be held in a rolled shape in the gap between the tip end portion 112 of the shaft member 11 and the cylindrical main body 122 that covers the tip end portion 112. More specifically, there is a distance of approximately 1 [mm], for example, between the outside surface of the tip end portion 112 and the inside surface of the cylindrical main body 122, and the rolled-up bio-implantable material F is arranged in this gap. Note that the distance between the outside surface of the tip end portion 112 and the inside surface of the cylindrical main body 122 is only an example and is not limited to this, and can be arbitrarily changed, as appropriate, in accordance with the thickness and modulus of rigidity and the like of the bio-implantable material F.

Also, when the tip end portion 112 around which the bio-implantable material F is wound is pulled out of the through-hole 23 of the sheet holder 2, at least one from among the shaft member 11 and the cylindrical member 12 is moved in the axial direction such that the cylindrical member 12 is displaced toward the tip end portion 112 side, and consequently, the tip end portion 112 becomes covered by the cylindrical main body 122.

That is, the outside diameter of the cylindrical main body 122 is smaller than the inside diameter of the large hole 23a of the through-hole 23, and larger than the inside diameter of the small hole 23b of the through-hole 23, so even if the cylindrical member 12 is inserted into the large hole 23a, the cylindrical member 12 will not be inserted into the small hole 23b. Also, after the bio-implantable material F is wound around the tip end portion 112 of the shaft member 11, for example, the cylindrical member 12 is displaced toward the tip end portion 112 side in response to an operation in which the tip end portion 112 is pulled out of the through-hole 23, such that the tip end portion 112 becomes covered by the cylindrical main body 122 of the cylindrical member 12 and the bio-implantable material F is held inside the cylindrical main body 122.

Examples of the operation in which the tip end portion 112 is pulled out of the through-hole 23 include an operation to move the sheet holder 2 toward the distal side in the axial direction while the position of the sheet winding device 1 is fixed, an operation to move the sheet winding device 1 toward the proximal side while the position of the sheet holder 2 is fixed, and an operation to move the sheet winding device 1 toward the proximal side while moving the sheet holder 2 toward the distal side in the axial direction, and the like.

<Method for Using the Sheet Winding System>

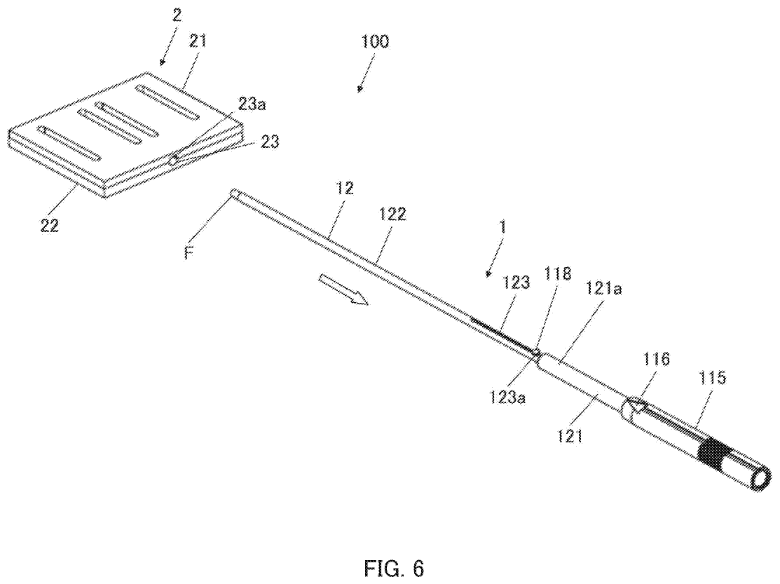

Next, the method for using the sheet winding system 100 will be described with reference to FIG. 1, FIG. 5, and FIG. 6.

FIG. 5 and FIG. 6 are views for explaining how to use the sheet winding system 100.

Note that in the following description, the bio-implantable material F that is used has a thickness of approximately 0.05 [mm] and a modulus of rigidity of approximately 50,000 [MPa].

<When Holding the Bio-implantable Material>

First, the sheet holder 2 into which the bio-implantable material F has been placed is prepared.

More specifically, after arranging the bio-implantable material F in the sheet arranging portion 222 of the lower holder structure member 22 of the sheet holder 2, the upper and lower holder structure members 21 and 22 are placed in an overlapped state by moving the upper holder structure member 21 toward the lower holder structure member 22 side. As a result, the bio-implantable material F is in a state held in the accommodating portion 24 (refer to FIG. 1).

Note that the bio-implantable material F is arranged so as to be substantially parallel to the left-right direction and the front-rear direction.

Also, the sheet winding device 1 in which the tip end portion 112 of the shaft member 11 is exposed is prepared.

More specifically, the tip end portion 112 is placed in a state (an exposed state) not covered by the cylindrical main body 122 of the cylindrical member 12, by moving the cylindrical member 12 toward the base end side while the position of the shaft member 11 is fixed.

Then, the user of the sheet winding device 1 aligns the orientation of the inserting slit 113 of the tip end portion 112 with the bio-implantable material F using the indicating portion 116 as a mark, and inserts the tip end portion 112 into the through-hole 23 of the sheet holder 2. At this time, the tip end portion 112 is inserted into the small hole 23b via the large hole 23a of the through-hole 23, so that the bio-implantable material F held in the accommodating portion 24 becomes inserted into the inserting slit 113, while the cylindrical member 12, although being inserted into the large hole 23a, becomes caught on the step portion between the large hole 23a and the small hole 23b (refer to FIG. 5).

Next, in the state in which the bio-implantable material F is inserted in the inserting slit 113, the shaft member 11 and the cylindrical member 12 are rotated together as a unit such that the tip end portion 112 rotates around its axis. As a result, the bio-implantable material F winds around the tip end portion 112.

Note that the sheet winding device 1 in a state in which the tip end portion 112 of the shaft member 11 is covered by the cylindrical main body 122 of the cylindrical member 12 may be used. In this case, when the tip end portion 112 that is covered by the cylindrical main body 122 is inserted into the through-hole 23 while the gripping portion 115 is being gripped by the user, the cylindrical main body 122 catches on the step portion between the large hole 23a and the small hole 23b such that displacement of the cylindrical main body 122 in the axial direction becomes restricted, and when the shaft member 11 is pushed in so as to move further toward the distal side from this state, the tip end portion 112 inserted into the small hole 23b while being exposed.

Then, at least one from among the shaft member 11 and the cylindrical member 12 is moved in the axial direction such that the cylindrical member 12 is displaced toward the tip end portion 112 side in response to an operation in which the tip end portion 112 is pulled out of the through-hole 23. As a result, the bio-implantable material F can be held in a rolled shape in the gap between the tip end portion 112 of the shaft member 11 and the cylindrical main body 122, with the tip end portion 112 covered by the cylindrical main body 122. Note that in FIG. 6, a portion on the tip end side of the bio-implantable material F is slightly exposed; as long as the majority of the bio-implantable material F is held in the gap between the tip end portion 112 and the cylindrical main body 122, it is not absolutely necessary that all of the bio-implantable material F be held.

Furthermore, the shaft portion of the protrusion 118 of the shaft main body 111 is engaged with the engaging portion 123a on the base end side of the retaining slit 123, so movement of the cylindrical member 12 along the axial direction is restricted. As a result, the cylindrical member 12 will not be displaced toward the base end side relative to the shaft member 11, so the hold on the rolled-up bio-implantable material F by the sheet winding device 1 will not be released.

<When Expanding the Bio-implantable Material>

First, the sheet winding device 1 in which the bio-implantable material F is held in a rolled form in the gap between the tip end portion 112 of the shaft member 11 and the cylindrical main body 122 is prepared.

Then, after making a position adjustment such that the tip end portion 112 of the shaft member 11 of the sheet winding device 1 is arranged in a holding position of the bio-implantable material F, the cylindrical member 12 is moved toward the base end side along the axial direction with respect to the shaft member 11. Then, the tip end portion 112 becomes exposed from the tip end side, and when the tip end portion 112 is completely exposed, the hold on the bio-implantable material F by the sheet winding device 1 is released, and the bio-implantable material F expands to the sheet-like shape.

As described above, the sheet winding system 100 of the present embodiment is a sheet winding system (bio-implantable material winding system) 100 provided with the sheet-like bio-implantable material F, and the sheet winding device 1 capable of winding the bio-implantable material F, in which the sheet winding device 1 is provided with the shaft member 11 and the cylindrical member 12 that fits over the shaft member 11 and is able to move in the axial direction relative to the shaft member 11, and the sheet winding device 1 is able to hold the bio-implantable material F in the rolled shape in the gap between the tip end portion 112 of the shaft member 11 and the cylindrical member 12 that covers the tip end portion 112, and the bio-implantable material F is able to expand to the sheet-like shape when the tip end portion 112 is exposed and the hold on the bio-implantable material F by the sheet winding device 1 is released.

Therefore, the bio-implantable material F can be appropriately held in a rolled shape by the sheet winding device 1, and when the hold is released, the bio-implantable material F naturally expands to the sheet-like shape, so the handleability of the sheet-like bio-implantable material F can be improved. In this way, it is possible to more easily apply the bio-implantable material F to a desired site by appropriately adjusting not only the structure of the sheet winding device 1 itself that holds the bio-implantable material F and releases the hold, but also the shape and physical properties such as the modulus of rigidity of the bio-implantable material F.

In particular, the bio-implantable material F held in the accommodating portion 24 is inserted into the inserting slit (inserting portion) 113 by the tip end portion 112 of the shaft member 11 of the sheet winding device 1 being inserted into the small hole (hole) 23b of the sheet holder (sheet accommodating device) 2, and the bio-implantable material F can be wound around the tip end portion 112 in a rolled shape by rotating the tip end portion 112 around its axis. Also, the tip end portion 112 is covered by the cylindrical member 12, and the bio-implantable material F can be held in a rolled shape in the gap between the tip end portion 112 and the cylindrical member 12, by moving at least one from among the shaft member 11 and the cylindrical member 12 in the axial direction such that the cylindrical member 12 is displaced toward the tip end portion 112 side around which the bio-implantable material F is wound.

Furthermore, moving at least one from among the shaft member 11 and the cylindrical member 12 in the axial direction such that the cylindrical member 12 is displaced toward the side opposite the tip end portion 112 around which the bio-implantable material F is wound causes the tip end portion 112 to become exposed, so the hold on the bio-implantable material F by the sheet winding device 1 can be released.

Note that the present invention is not limited to the embodiment described above; various improvements and design changes are possible without departing from the spirit of the present invention.

For example, in the embodiment described above, the cylindrical member 12 is displaced toward the tip end portion 112 side in response to an operation in which the tip end portion 112 is pulled out of through-hole 23, but this is only an example; the invention is not limited to this and it may be arbitrarily modified as appropriate. More specifically, by making the entire inside diameter of the through-hole 23 larger than the outside diameter of the cylindrical member 12, the cylindrical member 12 can be displaced toward the tip end portion 112 side and the bio-implantable material F can be held inside the cylindrical member 12 even in a state in which the tip end portion 112 of the shaft member 11 is inserted into the through-hole 23 and the bio-implantable material F is wound. In this case, the bio-implantable material F is pulled out from the sheet holder 2 while being held inside the cylindrical member 12.

Furthermore, a slit (not shown in the drawings) that extends in the axial direction corresponding to the orientation of the inserting slit 113 may also be provided in the tube wall of the cylindrical member 12. In this case, the bio-implantable material F can be wound around the tip end portion 112 and the bio-implantable material F can be held inside the cylindrical member 12, by inserting the tip end portion 112 into the through-hole 23 and rotating the tip end portion 112 its axis, while the tip end portion 112 is covered by the cylindrical member 12.

Furthermore, in the embodiment described above, the through-hole 23 is formed in the sheet holder 2, but this is only an example; the invention is not limited to this. Although not shown in the drawings, the hole may be a hole with a bottom.

Also, in the invention described above, the sheet holder 2 is given as an example of a part that is able to hold one sheet of bio-implantable material F, but the sheet holder 2 is only one example; the invention is not limited to this. For example, a plurality of accommodating portions 24 may be provided such that a plurality of sheets of the bio-implantable material F are able to be held.

Furthermore, the sheet winding system 100 of the present embodiment may also be applied to wound dressing material (not shown in the drawings) that covers a wound on the outside of the body, in addition to the bio-implantable material F that is inserted inside the body.

In addition, the embodiment disclosed herein is in all respects merely an example and should in no way be construed as limiting. The scope of the present invention is indicated not by the foregoing description but by the scope of the claims for patent, and is intended to include all modifications that are within the scope and meanings equivalent to the scope of the claims for patent.

The present application claims priority based on Japanese Patent Application No. 2017-163497 filed on Aug. 28, 2017. The contents described in the claims, specification, and drawings of the application are incorporated herein.

### REFERENCE SIGNS LIST

100 sheet winding system
1 sheet winding device
11 shaft member
111 shaft main body
112 tip end portion
113 inserting slit (inserting portion)
115 gripping portion
116 indicating portion
118 protrusion (external fitting release restricting portion)
12 cylindrical member
122 cylindrical main body
123 retaining slit (external fitting release restricting portion)
2 sheet holder (sheet accommodating device)
23 through-hole
23a large hole
23b small hole (hole)
24 accommodating portion
F bio-implantable material

1. A bio-implantable material winding system provided with sheet-like bio-implantable material, and a winding device capable of winding the bio-implantable material, wherein
the winding device comprising:
a shaft member, and
a cylindrical member that fits over the shaft member and is able to move in the axial direction relative to the shaft member, wherein the winding device is able to hold the bio-implantable material in a rolled shape in a gap between a tip end portion of the shaft member and the cylindrical member that covers the tip end portion, and
the bio-implantable material is able to expand to a sheet-like shape when the tip end portion is exposed and the hold by the winding device is released.

2. The bio-implantable material winding system according to claim 1, wherein
the bio-implantable material has a modulus of rigidity that enables the bio-implantable material to assume the rolled shape while being held by the winding device, and to expand to the sheet-like shape when the hold is released.

3. The bio-implantable material winding system according to claim 1, wherein
the tip end portion
has an inserting portion into which the bio-implantable material is able to be inserted, and is able to be inserted into a hole communicated with an accommodating portion of a sheet accommodating device, which accommodates the bio-implantable material, and
the bio-implantable material held in the accommodating portion is inserted into the inserting portion by inserting the tip end portion into the hole, and the bio-implantable material is wound in the rolled shape around the tip end portion by rotating the tip end portion around the axis thereof.

4. The bio-implantable material winding system according to claim 1, wherein
the tip end portion is covered by the cylindrical member, and the bio-implantable material is able to be held in the rolled shape in the gap between the tip end portion and the cylindrical member, by moving at least one from among the shaft member and the cylindrical member in the axial direction such that the cylindrical member is displaced toward the tip end portion side around which the bio-implantable material is wound.

5. The bio-implantable material winding system according to claim 1, wherein
the tip end portion is exposed and the hold on the bio-implantable material by the winding device is able to be released, by moving at least one from among the shaft member and the cylindrical member in the axial direction such that the cylindrical member is displaced toward the side opposite the tip end portion around which the bio-implantable material is wound.

6. The bio-implantable material winding system according to claim 1, further comprising:
an operation portion operated to displace the cylindrical member in the axial direction relative to the shaft member.
